(19) United States
(12) Patent Application Publication
(10) Pub. No.: US 2017/0100016 A1
(43) Pub. Date: Apr. 13, 2017

(54) CONTINUOUS FLOW SCOPE CONFIGURATION WITH OPTIONAL TOOL USAGE

(71) Applicant: COVIDIEN LP, MANSFIELD, MA (US)

(72) Inventors: NIKOLAI D. BEGG, WAYLAND, MA (US); DALIA LEIBOWITZ, WHITE PLAINS, NY (US); MIREILLE AKILIAN, SOMERVILLE, MA (US)

(21) Appl. No.: 15/286,815

(22) Filed: Oct. 6, 2016

Related U.S. Application Data
(60) Provisional application No. 62/240,113, filed on Oct. 12, 2015.
(60) Provisional application No. 62/257,413, filed on Nov. 19, 2015.

(51) Int. Cl. A61B 1/015; A61B 1/00; A61B 1/313; A61B 1/018

(52) U.S. Cl. CPC A61B 1/015; A61B 1/018; A61B 1/00126; A61B 1/313

(57) ABSTRACT

A continuous flow endoscope device includes an inflow and an outflow, an elongated tubular member, a first channel, a second channel, and an optics device. The elongated tubular member defines a lumen extending therethrough. The first channel extends through the lumen and is configured to receive an instrument therethrough. The second channel extends through the lumen. The optics device is disposed in a free space of the lumen not occupied by the first channel or the second channel. The first channel or the second channel is coupled to the inflow, and the other of the first channel or the second channel is coupled to the outflow.

### CROSS-REFERENCE TO RELATED APPLICATIONS

This application claims the benefit of U.S. Provisional Patent Application No. 62/240,113, filed on Oct. 12, 2015, and U.S. Provisional Patent Application No. 62/257,413, filed on Nov. 19, 2015, the entire contents of each of which are hereby incorporated herein by reference.

### BACKGROUND

Various types of endoscopes may be employed for surgical and exploratory procedures, some of which may involve fluid inflow, fluid outflow, or both, depending upon the operation being performed. Depending upon the operation being performed, there may be challenges to entry, access, and/or removal, or other challenges during and after the procedure, and the endoscope employed may need to adapt to these challenges by being configured for minimally invasive procedures or other procedures developed in response to those challenges.

### SUMMARY

A continuous flow endoscope device provided in accordance with the present disclosure includes an inflow and an outflow, an elongated tubular member defining a lumen extending therethrough, a first channel extending through the lumen and configured to receive an instrument therethrough, a second channel extending through the lumen, and an optics device disposed in a free space of the lumen not occupied by the first channel or the second channel. One of the first channel or the second channel is coupled to the inflow, and the other of the first channel or the second channel is coupled to the outflow.

In an aspect of the present disclosure, the first channel is coupled to the inflow and dedicated to fluid inflow, and the second channel is coupled to the outflow and dedicated to fluid outflow. Alternatively, the first channel is coupled to the outflow and dedicated to fluid outflow, and the second channel is coupled to the inflow and dedicated to fluid inflow.

In another aspect of the present disclosure, the device includes a body having an arm operably coupled to the optics device and adapted to connect to an imaging device.

In yet another aspect of the present disclosure, the first channel and the second channel define substantially similar fluid flow resistances with the instrument inserted through the first channel.

In still another aspect of the present disclosure, the first channel and the second channel define substantially similar fluid flow resistances in the absence of the instrument inserted through the first channel.

In still yet another aspect of the present disclosure, the first channel and the second channel define substantially similar cross-sectional fluid flow areas with the instrument inserted through the first channel.

In another aspect of the present disclosure, the endoscope device further includes at least one fluid control mechanism configured to maintain substantially similar fluid flow resistances through the first and second channels.

An endoscope system provided in accordance with the present disclosure includes a continuous flow endoscope device including an inflow and an outflow, an elongated tubular member defining a lumen extending therethrough, first and second channels extending through the lumen, and an optics device disposed in a free space of the lumen not occupied by the first channel or the second channel. One of the first channel or the second channel is coupled to the inflow, and the other of the first channel or the second channel is coupled to the outflow. The system further includes an instrument configured for insertion through the first channel and at least one fluid control mechanism operably associated with at least one of the inflow or the outflow.

In an aspect of the present disclosure, the system further includes a pump operably coupled to the inflow. One of the fluid control mechanism(s) may be incorporated into the pump.

In another aspect of the present disclosure, the system further includes an outflow reservoir operably coupled to the outflow. One of the fluid control mechanism(s) may be incorporated into the outflow reservoir.

In another aspect of the present disclosure, the first channel is coupled to the inflow and dedicated to fluid inflow, and the second channel is coupled to the outflow and dedicated to fluid outflow. Alternatively, the first channel is coupled to the outflow and dedicated to fluid outflow, and the second channel is coupled to the inflow and dedicated to fluid inflow.

In still another aspect of the present disclosure, the first channel and the second channel define substantially similar fluid flow resistances with the instrument inserted through the first channel.

In yet another aspect of the present disclosure, the first channel and the second channel define substantially similar fluid flow resistances in the absence of the instrument inserted through the first channel.

In still yet another aspect of the present disclosure, the first channel and the second channel define substantially similar cross-sectional fluid flow areas with the instrument inserted through the first channel.

In another aspect of the present disclosure, the at least one fluid control mechanism is configured to maintain substantially similar fluid flow resistances through the first and second channels.

A method provided in accordance with aspects of the present disclosure includes inserting an endoscope device into a surgical site. The endoscope device includes an elongated tubular member defining a lumen, a first channel extending through the lumen, a second channel extending through the lumen, and an optics device disposed in a free space of the lumen not occupied by the first channel or the second channel. The method further includes inserting an instrument through the first channel and into the surgical site, delivering fluid to the surgical site through one of the first channel or the second channel, removing fluid from the surgical site through the other of the first channel or the second channel, and maintaining continuous delivery and removal of fluid by maintaining substantially similar fluid flow resistances through the first and second channels.

### BRIEF DESCRIPTION OF THE DRAWINGS

A detailed description of the aspects and features of the present disclosure is provided below with reference to the accompanying drawings wherein:

FIG. 1 is a perspective view of a surgical system provided in accordance with the present disclosure including an endoscope device and a pump; and

FIG. 2 is a transverse, cross-sectional view of the endoscope device of FIG. 1 including an instrument inserted therethrough.

### DETAILED DESCRIPTION

Medical endoscope systems often contain channels in addition to their optical components in order to allow access for surgical instruments and fluid flow in and out of the operative field. In procedures conducted within a liquid environment, such as hysteroscopy, blood or other operative debris may cause impaired visualization. Visualization in a bloody or debris-filled liquid may be improved and maintained with continuous flow, because the constant circulation of fluid through the operative field continuously decreases the concentration of blood and/or debris. Continuous flow is achieved by providing separate pathways for fluid inflow and outflow from the operative field. In some cases, the addition of multiple channels within the endoscope creates a challenge with respect to maintaining an outer diameter of the endoscope small enough to provide atraumatic access to the operative field.

A number of endoscope designs may provide continuous flow as well as instrument access. These include endoscopes with three individual channels, one each for instrument access, inflow, and outflow, or an endoscope featuring a detachable outer sheath for outflow, where the endoscope contains either two individual lumens or one shared lumen for inflow and instrument access, and an outflow channel created by the annular space between the outer surface of the endoscope and the inner surface of the sheath. Endoscope designs may also feature a single channel shared by inflow and instrument access, and a removable outflow cannula which is inserted when there is no instrument in the channel. As a result, this design is not able to provide true continuous flow throughout the procedure. In currently employed devices when there is no instrument within the first channel, there is significantly more cross sectional area for flow in the first channel than in the second channel. As a result, the fluid resistance through the first channel will be significantly less than through the second channel. Since fluid flow rate is directly proportional to pressure difference and indirectly proportional to fluid resistance, it will take significantly greater pressure difference across the second channel to achieve the same fluid flow rate as through the first channel. If the first channel is used for fluid outflow during a procedure, fluid will flow out of the operative field with much less resistance than into the operative field, and it will likely be difficult to keep the operative field filled. This is especially true in procedures such as hysteroscopy where the operative field is pressurized to distend tissue and create space. The significant pressure difference across the outflow channel would make sufficient fluid inflow and pressure control within the cavity highly difficult to achieve.

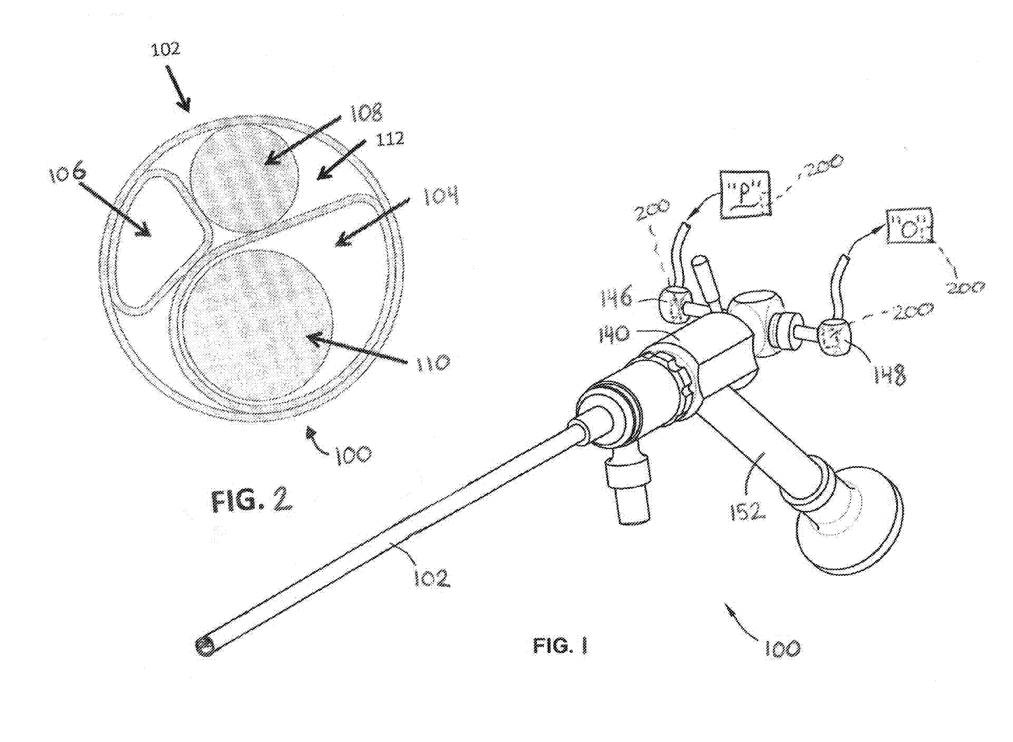

Referring generally to FIGS. 1 and 2, FIG. 1 is a perspective view of an endoscope device 100 provided in accordance with the present disclosure. Endoscope device 100 includes an elongated tubular member 102 and a proximal body 140. Proximal body 140 includes an inflow 146, an outflow 148, and an arm 152 that is connected to an imaging device (e.g., a camera) to capture images received via a visualization device, e.g., optics 108 (FIG. 2), extending through elongated tubular member 102. Endoscope device 100 forms a system in conjunction with pump “P” in communication with inflow 146 and/or an outflow reservoir “O” in communication with outflow 148. The system may be configured as an open system, wherein pump “P” and outflow reservoir “O” are separate, or may be a closed or partially-closed system, wherein outflow reservoir “O” is coupled to pump “P” or incorporated therein.

FIG. 2 is a transverse, cross-sectional view of the elongated tubular member 102 of endoscope device 100 (FIG. 1) including an instrument 110, e.g., a morcellator or other suitable surgical instrument, inserted therethrough. In some embodiments, elongated tubular member 102 defines a first channel 104 that is shared between fluid flow and instrument access, e.g., for instrument 110, and a second channel 106 for fluid flow as well. In some embodiments, the first channel 104 is shared between the instrument 110 and fluid outflow and, thus, is coupled to outflow 148 (FIG. 1), while the second channel 106 is employed for fluid inflow and, thus, is coupled to inflow 146 (FIG. 1). In other embodiments, the first channel 104 is shared between the instrument 110 and fluid inflow (and, thus, is coupled to inflow 146 (FIG. 1)), while the second channel 106 is employed for fluid outflow (and, thus, is coupled to outflow 148 (FIG. 1)).

Optics 108 extend through elongated tubular member 102 within a free space 112 thereof that is outside of the first and second channels 104, 106, respectively. This free space 112 may constitute any portion of the interior lumen defined by elongated tubular member 102 other than the portions occupied by the first and second channels 104,106, respectively.

Referring still to FIGS. 1 and 2, in embodiments of the present disclosure, the first and the second channels 104, 106, respectively, of the elongated tubular member 102 of the endoscope device 100 are configured both individually and relatively such that fluid resistance through the first channel 104 does not decrease to a level significantly lower than the fluid resistance through the second channel 106, regardless of whether an instrument 110 is inserted through first channel 104. “Significantly lower” and “significantly similar” as utilized herein may refer to when fluid flow through the first channel 104 is within a predetermined range of the fluid flow through the second channel 106. Other components of the endoscope device 100 and/or the system including the same are additionally or alternatively configured to maintain a substantially similar resistance between the first and second channels 104, 106, respectively, thus keeping the fluid flow between the channels 104, 106 substantially similar (within a predetermined range of each other).

In some embodiments, the cross-sectional areas accessible by fluid flow in both the first and second channels 104, 106, respectively, are relatively similar (with or without an instrument 110 inserted through one of the channels 104, 106). As a result, the fluid resistances created by the configuration of the first and second channels 104, 106, respectively, are substantially similar. The elongated tubular member 102 of the endoscope device 100 may include a variety of channel configurations and profile shapes, which allows for true continuous flow with or without an instrument 110 placed in the first channel 104, regardless of whether the first channel 104 is used for fluid outflow or fluid inflow. The determination as to whether the first channel 104 is used for fluid inflow or fluid outflow may be based on the type of procedure being performed, the patient, the involved medical professionals, and other factors that may impact the type and size of instrument(s) 110 employed in the procedure so that when the instrument(s) 110 are removed/replaced, fluid resistance is maintained between the first and second channels 104, 106, respectively.

The first and second channels 104, 106, respectively, may be configured to be different sizes and/or shapes (geometries), and may be permanently fixed within elongated tubular member 102. The elongated tubular member 102 also accommodates the optics device 108 while true continuous fluid flow is occurring in the first and second channels 104, 106, respectively, regardless of whether an instrument 110 is present in the first channel 104 or the second channel 106. The first channel 104 and/or the second channel 106 may taper the diameter of their respective cross-sections along the length of the elongated tubular member 102 in the proximal-to-distal direction. The first and second channels 104, 106, respectively, are not in communication with each other, e.g., are separate from one another. Further, no sheath is required for use with endoscope device 100.

The cross-section of the channel shared between fluid flow and instrument access, e.g., first channel 104, has an inner diameter greater than the outer diameter of instruments, e.g., instrument 110, inserted therethrough in order to enable flow in the resulting annular space. This shared channel 104 may also have a cross-section shaped differently from the outer profile of the instrument 110 in order to create additional space for fluid flow. As illustrated in FIG. 2, the cross-section of the shared channel 104 may, more specifically, include a portion that complements the outer profile of the instrument 110 and another portion that does not, e.g., extends away from, the outer profile of the instrument 110 to create the additional space for fluid flow.

The first and second channels 104, 106, respectively, may define a cross-sectional geometry of a circle, polygon, polygon with rounded edges, kidney, bean, teardrop, half-moon, triangle, and combinations thereof. The first and second channels 104, 106, respectively, may define different cross-sectional geometries or similar geometries, or similar geometries with different relative scales. The channels 104, 106 may be formed from rigid or semi-rigid biocompatible material and, as noted above, are disposed within the elongated tubular member 102 of endoscope device 100 without the aid of a sheath.

Instruments 110 used in conjunction with endoscope device 100 may be relatively large and take up a significant portion of the cross-section of the lumen of elongated tubular member 102 of endoscope device 100 when inserted through first channel 104. As a result, the first channel 104 may be relatively large and, together with the optics 108, may leave little space remaining for the second channel 106, which is, as a result, relatively small when compared with the first channel 104. When an instrument 110 is placed within the first channel 104, regardless of whether the first channel 104 is used for inflow or outflow, the cross-sectional areas accessible by fluid flow in both the first and second channels 104, 106 are substantially similar, so the fluid resistance of the first and second channels 104, 106 are similar, for example, when the length of the first channel 104 and the length of the second channel 106 are substantially similar (within a predetermined range of each other).

In some embodiments, the substantially similar resistance in the first and second channels 104, 106 may be achieved and/or maintained by an automatic, manual, electrical, mechanical, or electro-mechanical fluid control mechanism 200 of the pump “P” and/or the outflow reservoir “O.” Thus, the presence or absence of an instrument 110 within first channel 104 can be accounted for (or further accounted for).

In embodiments, the first channel 104 is used for fluid inflow and, thus, the fluid resistance of the second channel 106 (that has a smaller cross-section than the first channel 104) will be higher, allowing the operative field to remain pressurized. In embodiments where the first channel 104 is used for fluid inflow during a procedure, the fluid resistance of the fluid inflow will change throughout the procedure as instruments 110 are inserted into and removed from the endoscope device 100, since various instruments 110 may be employed throughout a procedure. In embodiments where the endoscope device 100 is used in combination with an external fluid control pump “P” (incorporating a fluid control mechanism 200 therein), the pump “P” will regulate the fluid inflow supply pressure in the first channel 104 in order to control the fluid pressure inside the operative space (e.g., the body cavity). In order to more accurately control pressure, the external fluid control pump “P” may employ the resistance of the fluid inflow path to calculate the theoretical pressure at the distal end of the elongated tubular member 102 of the endoscope device 100.

In some embodiments, the design of the channels 104, 106 may be sufficient to maintain the resistance of fluid flow. In some embodiments, if this measured resistance changes during the procedure and exceeds a predetermined amount, the fluid control mechanism 200 of the pump “P” is utilized to maintain the substantially similar resistances of fluid flow to control the pressure within the operative field to a desired accuracy. In some embodiments, the fluid control mechanism 200 of the pump “P” is not capable of maintaining such resistances. In these embodiments, an additional feature, such as another fluid control mechanism 200 associated with the outflow reservoir “O” coupled to the outflow 148, may be employed additionally or alternatively to maintain the fluid resistance of the fluid inflow path at a constant value.

In some embodiments, the first channel 104 is used for outflow, and the fluid resistance of the second (inflow) channel 106 does not change during the procedure, allowing the pump “P” to accurately control pressure because of the design of the respective channels 104, 106.

While exemplary embodiments of the invention have been shown and described, modifications thereof can be made by one skilled in the art without departing from the scope or teachings herein. The embodiments described herein are exemplary only and are not limiting. Many variations and modifications of the compositions, systems, apparatus, and processes described herein are possible and are within the scope of the invention. Accordingly, the scope of protection is not limited to the exemplary embodiments described herein, but is only limited by the claims that follow, the scope of which shall include all equivalents of the subject matter of the claims. Unless expressly stated otherwise, the steps in a method claim may be performed in any order and with any suitable combination of materials and processing conditions.

What is claimed is:

1. A continuous flow endoscope device, comprising:
an inflow;
an outflow;
an elongated tubular member defining a lumen extending therethrough;
a first channel extending through the lumen, the first channel configured to receive an instrument therethrough;
a second channel extending through the lumen; and
an optics device disposed in a free space of the lumen not occupied by the first channel or the second channel,
wherein, one of the first channel or the second channel is coupled to the inflow, and wherein the other of the first channel or the second channel is coupled to the outflow.

2. The device of claim 1, wherein the first channel is coupled to the inflow and dedicated to fluid inflow, and wherein the second channel is coupled to the outflow and dedicated to fluid outflow.

3. The device of claim 1, wherein the first channel is coupled to the outflow and dedicated to fluid outflow, and wherein the second channel is coupled to the inflow and dedicated to fluid inflow.

4. The device of claim 1, further comprising a body, wherein the elongated tubular member extends distally from the body, the body including an arm operably coupled to the optics device and adapted to connect to an imaging device.

5. The device of claim 1, wherein the first channel and the second channel define substantially similar fluid flow resistances with the instrument inserted through the first channel.

6. The device of claim 1, wherein the first channel and the second channel define substantially similar fluid flow resistances in the absence of the instrument inserted through the first channel.

7. The device of claim 1, wherein the first channel and the second channel define substantially similar cross-sectional fluid flow areas with the instrument inserted through the first channel.

8. The device of claim 1, further comprising at least one fluid control mechanism configured to maintain substantially similar fluid flow resistances through the first and second channels.

9. An endoscope system, comprising:
a continuous flow endoscope device, including:
an inflow;
an outflow;
an elongated tubular member defining a lumen extending therethrough;
a first channel extending through the lumen;
a second channel extending through the lumen, wherein one of the first channel or the second channel is coupled to the inflow, and wherein the other of the first channel or the second channel is coupled to the outflow; and
an optics device disposed in a free space of the lumen not occupied by the first channel or the second channel;
an instrument configured for insertion through the first channel; and
at least one fluid control mechanism operably associated with at least one of the inflow or the outflow.

10. The system of claim 9, further comprising a pump operably coupled to the inflow.

11. The system of claim 10, wherein one of the at least one fluid control mechanisms is incorporated into the pump.

12. The system of claim 9, further comprising an outflow reservoir operably coupled to the outflow.

13. The system of claim 12, wherein one of the at least one fluid control mechanisms is incorporated into the outflow reservoir.

14. The system of claim 9, wherein the first channel is coupled to the inflow and dedicated to fluid inflow, and wherein the second channel is coupled to the outflow and dedicated to fluid outflow.

15. The system of claim 9, wherein the first channel is coupled to the outflow and dedicated to fluid outflow, and wherein the second channel is coupled to the inflow and dedicated to fluid inflow.

16. The system of claim 9, wherein the first channel and the second channel define substantially similar fluid flow resistances with the instrument inserted through the first channel.

17. The system of claim 9, wherein the first channel and the second channel define substantially similar fluid flow resistances in the absence of the instrument inserted through the first channel.

18. The system of claim 9, wherein the first channel and the second channel define substantially similar cross-sectional fluid flow areas with the instrument inserted through the first channel.

19. The system of claim 9, wherein the at least one fluid control mechanism is configured to maintain substantially similar fluid flow resistances through the first and second channels.

20. A method, comprising:
inserting an endoscope device into a surgical site, the endoscope device including an elongated tubular member defining a lumen, a first channel extending through the lumen, a second channel extending through the lumen, and an optics device disposed in a free space of the lumen not occupied by the first channel or the second channel;
inserting an instrument through the first channel and into the surgical site;
delivering fluid to the surgical site through one of the first channel or the second channel;
removing fluid from the surgical site through the other of the first channel or the second channel; and
maintaining continuous delivery and removal of fluid by maintaining substantially similar fluid flow resistances through the first and second channels.
